(19) United States
(12) Patent Application Publication
(10) Pub. No.: US 2023/0235263 A1
(43) Pub. Date: Jul. 27, 2023

(54) SYSTEMS AND METHODS FOR FILTRATION OF CELL CULTURES

(71) Applicant: Repligen Corporation, Waltham, MA (US)

(72) Inventors: Shashi Kudugunti, Windham, NH (US); Earl Pineda, Anglet (FR); Michael Bransby, Altadena, CA (US)

(73) Assignee: Repligen Corporation, Waltham, MA (US)

(21) Appl. No.: 18/098,926

(22) Filed: Jan. 19, 2023

Related U.S. Application Data
(60) Provisional application No. 63/301,782, filed on Jan. 21, 2022.

(51) Int. Cl. C12M 1/00; C12M 1/26

(52) U.S. Cl. CPC C12M 29/10; C12M 29/04; C12M 33/14; C12M 47/04

(57) ABSTRACT

A method of improving volumetric productivity from a cell culture includes filtering a cell culture through an ultrafilter or a microfilter operating in tangential flow filtration mode or alternating flow filtration mode, and filtering the cell culture concurrently or intermittently through a tangential flow depth filtration system to remove cellular debris and/or to harvest cell product. A system for filtering biological materials includes a primary filtration system, and a secondary filtration system, where the secondary filtration system comprises a tangential flow depth filtration filter.

### CROSS-REFERENCE TO RELATED APPLICATIONS

This is a non-provisional of pending U.S. Provisional Patent Application Ser. No. 63/301,782, filed Jan. 21, 2022, titled “Systems and Methods for Filtration of Cell Cultures,” the entirety of which is incorporated by reference herein.

### FIELD

The present disclosure relates generally to the field of filtration. In particular, the present disclosure relates to filtration of fluids, particularly biological fluids, and even more particularly cell cultures, such as for biotechnology and/or pharmaceutical industries. More particularly, the present disclosure relates to improved systems and methods of filtration using tangential flow depth filtration.

### BACKGROUND

Cultures of living cells (e.g., microbial, plant, or animal cells) suspended in growth media, for example in a bioreactor, have been used to produce biological and chemical substances of significant commercial value, such as for pharmaceutical use. Applications include fermentation, biotechnology, and chemical, for production of specialty chemicals and products, as well as waste-treatment. The products are typically high-value products that include any desired cellular products, such as endogenous and recombinant products, including proteins, peptides, nucleic acids, virus, amino acids, antibiotics, specialty chemicals, and other molecules of value. Desired proteins may include but are not limited to monoclonal antibodies, enzymes and other recombinant antibodies, enzymes, peptides, virus, etc. Even marginal improvements in yield and productivity increase profitability. Particularly for commercial production, cell cultures can be run in a variety of operational modes, such as batch, continuous, fed-batch, concentrated fed-batch, or perfusion.

In normal batch culture production processes, cells inoculated into fresh medium rapidly enter a logarithmic growth phase. As they consume the media, nutrients and waste products accumulate, and the cells transition to a stationary phase followed by a decay phase. While several methods have been developed to optimize batch culture production, in each cycle, cells undergo rapid growth and decay phases. Fed batch culture refers to an operational technique for biotechnological processes where one or more nutrients necessary for cell growth and product formation are fed or supplied to the bioreactor during cultivation either intermittently or continuously via one or more feed streams during the course of an otherwise batch operation. There are no effluent streams during the course of operation, so the bioreactor products remain in the bioreactor until the end of the run when harvesting begins. This process may be repeated a number of times if the cells are fully viable and productive. Fed-batch cultures are advantageous since the fed-batch operation can provide unique means of regulating the concentration of compounds that control key reaction rates and, therefore, can provide a definite advantage over the batch operation through the manipulation of one or more feed rates. Fed-batch culture is also advantageous for large-scale production due to its operational simplicity and familiarity as a carryover process from fermentation.

Concentrated fed-batch (CFB) uses a perfusion system with an ultrafiltration membrane (e.g., 50 kDa or 30 kDa nominal molecular weight cutoff) that retains the protein product in the bioreactor while removing waste products and feeding additional media into the reactor vessel. This process obtains higher cell concentration but retains product in the reactor like a conventional fed batch process.

Unlike the constant changing conditions during batch culture method of production, the perfusion method offers a way to achieve and maintain a culture in steady state. In the perfusion method for growing cells, culture media, whose nutrients have been consumed and which contains increased levels of harmful waste products, are continuously removed and replaced with fresh media. The constant addition of fresh media while eliminating waste products provides the cells with the nutrients it requires to achieve high cell concentrations. Since waste products generated by the culture are continuously removed and the culture is continuously replenished with fresh media, it is possible to achieve a state of equilibrium in which cell concentration and productivity are maintained. Typically, about one culture volume is exchanged per day and the cell concentration achieved in perfusion are typically 2 to more than 10 times that achieved at the peak of batch culture.

There are incentives to improve the various cell culture production processes. Filtration is typically performed to separate, clarify, modify and/or concentrate a fluid solution, mixture, or suspension. In the biotechnology and pharmaceutical industries, filtration is vital for the successful production, processing, and testing of new drugs, diagnostics, and other biological products. In the process of manufacturing biologicals, such as using animal or microbial cell culture, filtration is used for clarification, selective removal, and concentration of certain constituents from the culture media, or to modify the media prior to further processing. Filtration may also be used to enhance productivity by maintaining a culture in perfusion at high cell concentration. Molecules produced by the cells may be secreted into the growth medium for collection therefrom, such as via filtration. Additionally or alternatively, the product to be harvested (e.g., with the use of a filter) may be within the cells or may constitute the cells (or virus particles) themselves. In some instances, filters are used to remove waste products rather than to harvest cellular products or other desired materials.

As may be appreciated, improvements to filtration processes would be welcome in the industry to improve cell culture production processes.

### SUMMARY

This summary of the disclosure is given to aid understanding, and one of skill in the art will understand that each of the various aspects and features of the disclosure may advantageously be used separately in some instances, or in combination with other aspects and features of the disclosure in other instances. No limitation as to the scope of the claimed subject matter is intended by either the inclusion or non-inclusion of elements, components, or the like in this summary.

In accordance with various principles of the present disclosure, a method of improving volumetric productivity from a cell culture includes filtering a cell culture through an ultrafilter or a microfilter operating in tangential flow filtration mode or alternating flow filtration mode; and filtering the cell culture concurrently or intermittently through a tangential flow depth filtration system to remove cellular debris and/or to harvest cell product.

In some embodiments, the cell culture is a perfusion or concentrated fed-batch cell culture. In some embodiments, filtering the cell culture through an ultrafilter or microfilter includes using a microfilter operating in tangential flow filtration mode or alternating tangential filtration mode. In some embodiments, filtering the cell culture through an ultrafilter or microfilter includes using an ultrafilter operating in tangential flow filtration or alternating tangential filtration mode. In some embodiments, the cell culture is a concentrated fed-batch cell culture, and filtering the cell culture uses an ultrafilter operating in tangential flow filtration or alternating tangential filtration mode.

In one aspect, filtering the cell culture through a tangential flow depth filtration system prolongs the usable life of the ultrafilter or microfilter.

In accordance with various principles of the present disclosure, a system for filtering biological materials includes a primary filtration system, and a secondary filtration system, the secondary filtration system including a tangential flow depth filtration filter.

In some embodiments, the primary filtration system includes a microfilter or an ultrafilter.

In some embodiments, the system for filtering biological materials produces a cell culture utilizing a batch production process.

In some embodiments, the primary filtration system is on continuously and the secondary filtration system is turned on continuously or intermittently. The cell culture may be produced utilizing a batch production process.

In some embodiments, the system for filtering biological materials produces a cell culture utilizing a continuous production process. In some embodiments, greater than 50% of the volume of the cell culture is filtered by the primary filtration system and less than 50% of the volume of the cell culture is filtered by the secondary filtration system. In some embodiments, 80% of the volume of the cell culture is filtered by the primary filtration system and 20% of the volume of the cell culture is filtered by the secondary filtration system.

In some embodiments, the primary filtration system includes a microfilter and the system for filtering biological materials produces a cell culture utilizing a batch production process.

In some embodiments, the primary filtration system includes a microfilter and the system for filtering biological materials produces a cell culture utilizing a continuous production process.

In some embodiments, the primary filtration system includes an ultrafilter and the system for filtering biological materials produces a cell culture utilizing a batch production process.

In some embodiments, the primary filtration system is on continuously and the secondary filtration system is turned on intermittently.

In some embodiments, the flow volume to the secondary filtration system is less than the flow volume to the primary filtration system.

In some embodiments, the primary filtration system is an alternating tangential flow filtration system. The cell culture may be a perfusion or concentrated fed-batch cell culture.

In some embodiments, the system also includes a process vessel.

In accordance with various principles of the present disclosure, a method of harvesting a cell product from a cell culture includes (a) harvesting the cell product from a perfusion or concentrated fed-batch cell culture using a microfilter operating in tangential flow filtration or alternating tangential flow mode into a first permeate; (b) removing cellular debris and further harvesting the cell product by concurrently or intermittently filtering the cell culture using a tangential flow depth filtration filter operating in tangential flow filtration or alternating tangential flow mode into a second permeate, and (c) recovering the cell product from the first and second permeates. In some embodiments, the cell culture is a perfusion or concentrated fed-batch cell culture.

In some embodiments, one or both of steps (a) and (b) are independently operated in alternating tangential filtration mode.

In accordance with various principles of the present disclosure, a method of harvesting a cell product from a cell culture includes (a) perfusing the media of a concentrated fed-batch cell culture using an ultrafilter operating in tangential flow filtration or alternating tangential flow mode to remove spent media; (b) removing cellular debris and further harvesting the cell product by concurrently or intermittently filtering the cell culture using a tangential flow depth filtration filter operating in tangential flow filtration or alternating tangential flow mode into a permeate, and (c) recovering the cell product from the permeate. In some embodiments, the cell culture is a concentrated fed-batch cell culture.

In some embodiments, one or both of steps (a) and (b) are independently operated in alternating tangential filtration mode.

In one aspect, the cell product of methods of the present disclosure may be a protein or a viral particle.

In one aspect, the overall production yield of cell product per production lifetime of the cell culture in accordance with various principles of the present disclosure is greater than that which would have been obtained by a conventional harvesting method.

These and other features and advantages of the present disclosure, will be readily apparent from the following detailed description, the scope of the claimed invention being set out in the appended claims. While the following disclosure is presented in terms of aspects or embodiments, it should be appreciated that individual aspects can be claimed separately or in combination with aspects and features of that embodiment or any other embodiment.

### BRIEF DESCRIPTION OF THE DRAWINGS

Non-limiting embodiments of the present disclosure are described by way of example with reference to the accompanying drawings, which are schematic and not intended to be drawn to scale. The accompanying drawings are provided for purposes of illustration only, and the dimensions, positions, order, and relative sizes reflected in the figures in the drawings may vary. For example, devices may be enlarged so that detail is discernable, but is intended to be scaled down in relation to another component. For purposes of clarity and simplicity, not every element is labeled in every figure, nor is every element of each embodiment shown where illustration is not necessary to allow those of ordinary skill in the art to understand the disclosure.

The detailed description will be better understood in conjunction with the accompanying drawings, wherein like reference characters represent like elements, as follows:

FIG. 1 illustrates a schematic representation of a dual filtration system formed in accordance with various principles of the present disclosure.

FIG. 2 illustrates a schematic representation of another dual filtration system formed in accordance with various principles of the present disclosure

FIG. 3 is a schematic cross-sectional view of a wall of a tangential flow depth filter which may be used in a system as in FIG. 1 or FIG. 2.

### DETAILED DESCRIPTION

The following detailed description should be read with reference to the drawings, which depict illustrative embodiments. It is to be understood that the disclosure is not limited to the particular embodiments described, as such may vary. All apparatuses and systems and methods discussed herein are examples of apparatuses and/or systems and/or methods implemented in accordance with one or more principles of this disclosure. Each example of an embodiment is provided by way of explanation and is not the only way to implement these principles but are merely examples. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure. In fact, it will be apparent to those skilled in the art that various modifications and variations can be made in the present disclosure without departing from the scope or spirit of the present subject matter. For instance, features illustrated or described as part of one embodiment can be used with another embodiment to yield a still further embodiment. Thus, it is intended that the present subject matter covers such modifications and variations as come within the scope of the appended claims and their equivalents.

It will be appreciated that the present disclosure is set forth in various levels of detail in this application. In certain instances, details that are not necessary for one of ordinary skill in the art to understand the disclosure, or that render other details difficult to perceive may have been omitted. The terminology used herein is for the purpose of describing particular embodiments only, and is not intended to be limiting beyond the scope of the appended claims. Unless defined otherwise, technical terms used herein are to be understood as commonly understood by one of ordinary skill in the art to which the disclosure belongs. All of the devices and/or methods disclosed and claimed herein can be made and executed without undue experimentation in light of the present disclosure.

As used herein, fluid flow refers to a supernatant or media along with various materials suspended therein, including, without limitation, “large” particles such as cells; “intermediate-sized” particles such as cellular debris; and “small” particles such as soluble and insoluble cell metabolites and other products produced by cells including, without limitation, expressed proteins, viruses, virus like particles (VLP's), exosomes, lipids, DNA, other metabolites, etc. It will be appreciated that the term fluid flow may be used interchangeably herein with terms such as feed stream, feed material, inlet flow, cell culture, culture medium, culture broth, etc., without intent to limit.

References are made herein to a cell culture grown in a process vessel. The process vessel may be any suitable container for housing a fluid to be filtered. For example, the process vessel may be a bioreactor, a fermentor, or any other vessel, including, without limitation, vats, barrels, tanks, bottles, flasks, containers, and the like which can contain liquids. The process vessel may be composed of any suitable material such as plastic, metal such as stainless steel, glass, or the like. One or more appropriate fluid connectors (piping, tubing, couplings, valves, etc.) can be used to fluidly couple the process vessel to a pump and/or filtration system.

Reference is made herein to tangential flow filtration (also referred to as cross-flow filtration or TFF) systems. Such systems are used in the separation of particulates suspended in a liquid phase, and have important bioprocessing applications. In contrast with depth filtration systems, in which a single fluid feed is passed through a filter in a direction transverse to the filter, tangential flow systems are characterized by fluid feeds that flow along (in other words, generally tangential to, in contrast with transverse and through) a surface of the filter, resulting in the separation of the feed into two components: a permeate component which has passed through the filter, and a retentate component which continues to flow along the filter without passing through the filter. Compared to depth filtration systems, TFF systems are less prone to fouling. Additionally, TFF systems generally are gentler on the cells carried by the fluid flow along the filter than other filtration systems.

Fouling of filtration systems using tangential rather than depth flow may be reduced further by alternating (generally, regularly/cyclically) the direction of the fluid feed across the filtration element as is done in the XCell ATF® alternating tangential flow system sold by Repligen Corporation (Waltham, Mass.), such as by backwashing the permeate through the filter, and/or by periodic washing. Alternating tangential flow (“ATF”) filtration systems circulate fluid flow from a vessel (e.g., a bioreactor) across a filter in a first direction, and then back across the filter in a second direction opposite the first direction to return to the vessel. One or more of a variety of pumps may be used, such as, without limitation, diaphragm pumps, peristaltic pumps, piston pumps, etc., to circulate the fluid across the filter. A relatively uniform flow can be produced across the entire filter. Moreover, the fluid flow may be relatively rapid, yet with a low shear force, thus sieving desired materials through the filter without damaging the materials.

Various specialized filters and filtration methods are available for separating materials according to their chemical and/or physical properties, such as flat surface filters, pleated filters, multi-unit cassettes, and tubular filters such as hollow fiber filters. However, many of these filters have short operating lives (relative to the term of the cell culture process), and when used to filter cell culture suspension or other biological fluids, filters tend to clog with dead cells, cell debris, aggregates, or other constituents of the fluid. Tangential flow depth filtration (“TFDF”) filters have been developed to address fouling issues associated with prior art filters. Like prior hollow fiber filters, TFDF filters may be generally tubular hollow fibers through which fluid is filtered by passing through the interior lumen of the TFDF filter and through the walls thereof. However, in contrast with standard thin wall hollow fiber filters which may accumulate a protein and cell debris fouling gel layer when using tangential flow filtration, the wall of a TFDF filter is thicker (e.g., at least about 1 mm thick and up to about 10 mm thick) and has nonuniform pores therethrough. As such, the wall of TFDF filters adds what is referred to herein as a depth filtration feature that traps the cell debris inside the wall structure, enabling increased volumetric throughput while maintaining close to 100% passage of typical target proteins. In particular, the wall of an TFDF filter includes tortuous paths that capture certain elements (i.e., intermediate-sized particles) of the fluid flow as a portion of the fluid flow passes through the wall of hollow fiber TFDF filter while allowing other particles (i.e., small particles) to pass through the wall as part of the permeate flow. Settling zones and narrowing channels may be formed in the wall to capture intermediate-size particles which enter the tortuous paths, while allowing smaller particles to pass through the wall, thus trapping intermediate-size particles and causing a separation of the intermediate-size particles from smaller particles in the permeate flow. This method is thus different from filtering obtained by the surface of standard thin wall hollow fiber tangential flow filter membranes, wherein intermediate-size particles can build up at the inner surface of the wall, clogging entrances to the tortuous paths. Moreover, TFDF filters may have smaller inner diameters (e.g., as small as approximately 0.75 mm) than the inner diameter of standard hollow fiber filters, which may result in an increase in shear rate which may enhance flushing of cells and cell debris from the inner wall of the hollow interior of the TFDF filter. The pore structure of TFDF filters generally is not precisely defined. Particles that are larger than the “pore size” of the filter will be stopped at the surface of the filter. A significant quantity of intermediate-sized particles, on the other hand, enter the wall of the filter and become entrapped within the wall. Smaller particles and soluble materials can pass though the filter material in the permeate flow. Being of thicker construction and higher porosity than many other filters in the art, the filters can exhibit enhanced flow rates and what is known in the filtration art as “dirt loading capacity,” which is the quantity of particulate matter a filter can trap and hold before a maximum allowable back pressure is reached.

In accordance with various principles of the present disclosure, a cell culture production system and method (e.g., batch mode, continuous mode, etc.) having a first filtration system is improved and optimized by the addition of a second filtration system, which may be considered a supplemental filtration system. More particularly, various filtration configurations are used to filter one or more components or products from a cell culture through a primary filtration system. In some aspects, the present disclosure utilizes tangential flow filtration across different filtration systems. In some aspects, the present disclosure utilizes an alternating tangential flow across different filtration systems. The cell culture may generally flow through the primary filtration system on a substantially continuous basis. In accordance with various principles of the present disclosure, a secondary filtration system is provided for selective filtration, such as intermittent filtration (at selected times during the process, rather than continuously) or partial filtration (only a portion of harvest flow being processed or filtered). Such secondary filtration may be considered to supplement the primary filtration system or to provide different filtration functions. Provision of a secondary filtration system, such as a TFDF system, has been found to extend the usable life of the primary filtration system, such as by filtering products which may tend to foul the primary filtration system (thereby extending the time before the primary filtration system fouls from such products); by periodic harvesting while the primary filtration system primarily acts to maintain healthiness of the culture (such as by filtering out undesired materials, by-products, waste, etc., and/or for media exchange); by supplementing harvesting performed by the primary filtration system to improve sieving rates and production; and other benefits. For instance, the secondary filtration system may remove/entrap cell debris, reducing the burden on the filter (e.g., a 0.2 μm filter) of the primary filtration system.

Generally, TFDF systems are primarily used as a clarification tool for fed-batch cultures. In accordance with various principles of the present disclosure, the TFDF system is used in a new manner, as a completely new tool, i.e., “cell debris entrapment/removal” for batch or continuous cell culture operations. The membrane design, morphology, and pore structure of TFDF systems greatly help to either trap the cell debris or remove the cell debris reducing the foulants concentrations in the bioreactors. Literature suggests that cell debris is one of the primary factors for ATF 0.2 μm perfusion hollow fiber membrane fouling. Hence, combing these two technologies—ATF, TFDF as a secondary system—to trap cell debris in membrane pores reduces the burden on the primary filter which, in turn, prolongs the filter life and the productivity of the overall process.

A filtration system and method in accordance with various principles of the present disclosure has one or more primary filtrations systems. The “primary” filtration system has a filter selected based on the type of filtration desired. For instance, the filter of the primary filtration system is selected based on the product desired to be obtained from the fluid flow and the further processing needed for that product. The filter can additionally or alternatively be selected based on the retentate desired to be returned to the fluid vessel from which fluid is obtained (e.g., a bioreactor such as in the case of a cell culture). For example, a microfilter (such as with pores having a diameter of approximately 0.2 μm-0.65 μm and/or a pore size of approximately 750 kD-0.65 kD) is suitable for harvesting a fed batch culture because a microfilter retains cells and also other intermediate size particulates (e.g., cell debris), which, if removed, are beneficial for downstream processes However, such filter generally retains the cell debris, which leads to a faster filter fouling. Use of an additional secondary filter (e.g., TFDF), in accordance with various principles of the present disclosure, removes the cell debris and lessens the rate of filter fouling. A microfilter may be used in various applications, including steady-state perfusion, dynamic perfusion, and (n−1) perfusion where the protein product is harvested through the filter permeate. An ultrafilter (such as with a nominal pore size of approximately 50 kD and as small as approximately 10 kD, and as large as approximately 500 kD for larger species would be suitable to remove waste materials while retaining the large biological products (e.g., proteins, viruses, cells and the like) within the culture medium. For instance, an ultrafilter may retain the protein product and allow only waste components to permeate therethrough. Typically, an ultrafilter may be used in concentrated fed-batch applications. The filters may be in the form of hollow fiber filters, such as known to those of ordinary skill in the art.

Additionally, in accordance with various principles of the present disclosure, TFDF is used in a secondary filtration system in conjunction with the primary filtration system to result in various benefits such as, longer usability of the primary filtration system by removing the foulants (cell debris or small particles), improving the culture environment, higher volumetric throughput (L/in²), better sieving profiles, etc. Depth filtration, such as with TFDF filters, may be particularly effective in the systems formed in accordance with various principles of the present disclosure when the fluid to be filtered contains a high load of particles as such filters can retain a large mass of particles before becoming clogged compared to other types of filters. TFDF filtration may be typified by multiple porous layers whose depth are used to capture solid components (e.g., contaminants) from a fluid stream.

In accordance with various principles of the present disclosure, TFDF may be used to optimize batch mode or continuous mode cell culture operations. The TFDF system may be configured for removing cell debris or to harvest desired materials from the cell culture, depending on the function of the primary filtration system used with the cell culture. The TFDF filtration system thus functions, in accordance with various principles of the present disclosure, in a secondary filtration system in conjunction with the primary filtration system to optimize cell production and/or harvesting of desired biological products.

A secondary filtration system (e.g., TFDF) may be used in accordance with various principles of the present disclosure intermittently and/or for only a portion of the fluid flow through the cell culture system. For instance, in some embodiments, the primary filtration system is used substantially continuously, whereas the secondary filtration system is used only a portion of the time the primary filtration system is in use. Additionally or alternatively, only a portion of the total volume of fluid flow in a cell culture system may be passed through the secondary filtration system during operation of the cell culture system. For instance, less than half the total volume of fluid flow through the cell culture system may be filtered through the secondary filtration system while the remainder (majority) of the fluid flow passes through the primary filtration system.

Various embodiments of filtration systems formed in accordance with various principles of the present disclosure will now be described with reference to the examples of embodiments illustrated in the accompanying drawings. Reference in this specification to “one embodiment,” “an embodiment,” “some embodiments”, “other embodiments”, etc. indicates that one or more particular features, structures, and/or characteristics in accordance with principles of the present disclosure may be included in connection with the embodiment. However, such references do not necessarily mean that all embodiments include the particular features, structures, and/or characteristics, or that an embodiment includes all features, structures, and/or characteristics. Some embodiments may include one or more such features, structures, and/or characteristics, in various combinations thereof. Moreover, references to “one embodiment,” “an embodiment,” “some embodiments”, “other embodiments”, etc. in various places in the specification are not necessarily all referring to the same embodiment, nor are separate or alternative embodiments necessarily mutually exclusive of other embodiments. When particular features, structures, and/or characteristics are described in connection with one embodiment, it should be understood that such features, structures, and/or characteristics may also be used in connection with other embodiments whether or not explicitly described, unless clearly stated to the contrary. It should further be understood that such features, structures, and/or characteristics may be used or present singly or in various combinations with one another to create alternative embodiments which are considered part of the present disclosure, as it would be too cumbersome to describe all of the numerous possible combinations and subcombinations of features, structures, and/or characteristics. Moreover, various features, structures, and/or characteristics are described which may be exhibited by some embodiments and not by others. Similarly, various features, structures, and/or characteristics or requirements are described which may be features, structures, and/or characteristics or requirements for some embodiments but may not be features, structures, and/or characteristics or requirements for other embodiments. Therefore, the present disclosure is not limited to only the embodiments specifically described herein.

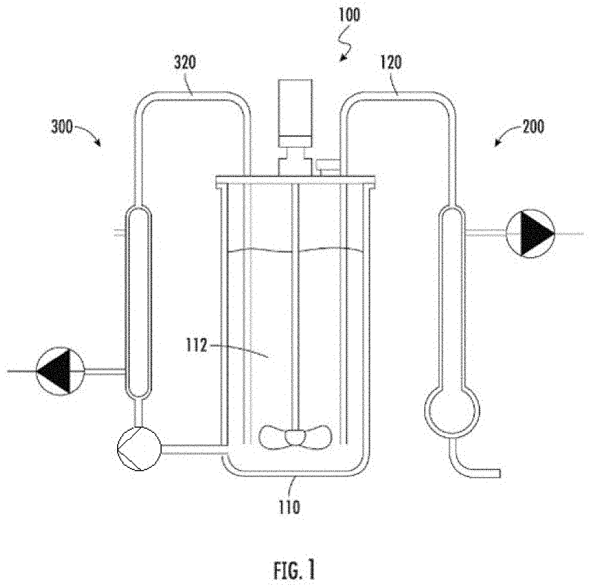

Turning now to the drawings, a schematic representation of a cell culture system 100 formed in accordance with various principles of the present disclosure is illustrated in FIG. 1. The illustrated cell culture system 100 includes a process vessel 110 in which a cell culture 112 is grown, and a primary filtration system 200 for harvesting cellular products (e.g., endogenous and recombinant products, including (target) proteins, peptides, nucleic acids, virus, amino acids, antibiotics, specialty chemicals, and other molecules of value) and/or for removing waste material(s) (e.g., cellular waste products such as lactic acid, ammonia, lactose, hydrogen, ethanol, etc., or cellular debris). The process vessel 110 is fluidly coupled with the primary filtration system 200 via at least one primary filtration feed line 120. Depending on the type of cell culture process being used, the cell culture system 100 may include additional fluid flow lines coupled to the process vessel 110 (e.g., to feed media, additives, additional cells, etc. into the process vessel 110 and/or to remove other materials), and optional accompanying components (e.g., pumps, valves, etc.) in a manner known to those of ordinary skill in the art.

In accordance with various principles of the present disclosure, any of a variety of culture production processes, including, without limitation, batch, continuous, fed-batch, or concentrated fed-batch, may be performed with or within a cell culture system 100. The primary filtration system 200 of a cell culture system 100 utilizes a filter selected based on the desired filtration to be achieved (such as in view of the content of the permeate which is to pass through the filter). For instance, a microfilter (e.g., with a pore size of approximately 750 kD-0.65 kD) may be used to remove (harvest) cellular products (e.g., proteins) for further processing, or for media exchange or waste removal. Alternatively, an ultrafilter (with a pore size of approximately 10 kD-50 kD) may be used for removal of waste products or unwanted cellular by-products or spent media. Selection of a filter for the primary filtration system 200 may also be influenced by the culture production process as may be appreciated by one of ordinary skill in the art. Commonly, the filter has pores with a diameter of approximately 0.2 μm. Regardless of pore size, the filter gets fouled by cell debris which interferes with the desired functioning of the system.

In accordance with various principles of the present disclosure, a cell culture system 100 is provided with a secondary filtration system 300 which functions in conjunction with the primary filtration system 200. Fluid flow may be directed from the process vessel 110 to the secondary filtration system 300 via a secondary filtration feed line 320. The secondary filtration system 300 may optimize performance of the primary filtration system 200 and thus performance of the cell culture system 100 in a variety of manners. For instance, the secondary filtration system 300 may help remove cell debris and increase protein sieving. Additionally or alternatively, the secondary filtration system 300 may split the harvest (amount or rate of sieving) with the primary filtration system 200 to increase overall sieving capability of the filtration system 100. In general, the secondary filtration system 300 may be used to enhance the culture conditions by removal of cell debris and/or by harvesting proteins at optimal times during cell growth in the culture.

In one example of an embodiment of a cell culture system 100 run in batch mode with a primary filtration system 200 and a secondary filtration system 300, the primary filtration system 200 includes a microfilter. The microfilter may be used to harvest proteins produced by the cells in the cell culture 112. The microfilter has a filtration capacity of approximately 750 kD or 0.65 μm. In some aspects, the primary filtration system 200 may be a ATF system, while the secondary filtration system 300 is a TFDF system that includes a TFDF filter.

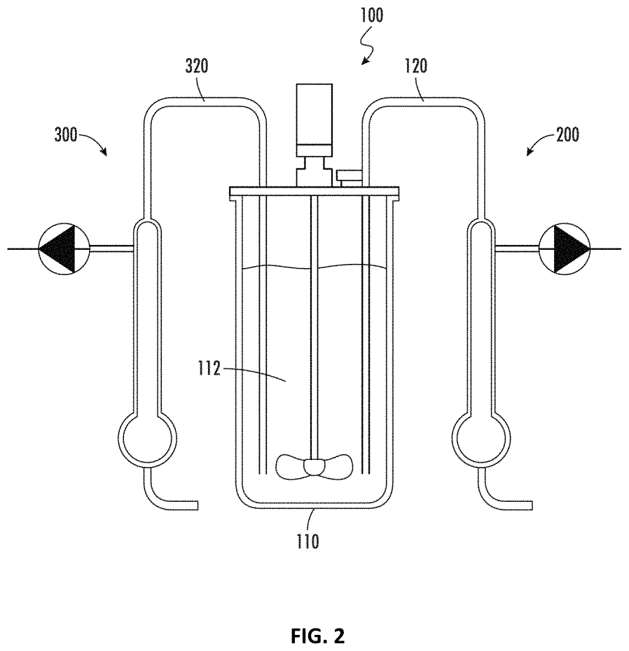

In some embodiments, such as the arrangement illustrated in FIG. 2, the primary and secondary filtration systems 200, 300 are ATF systems.

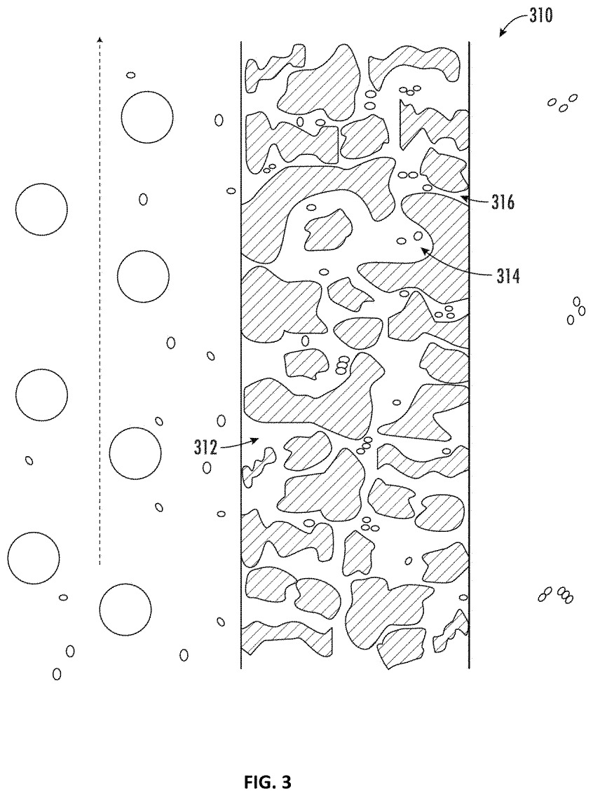

Referring to FIG. 3, a portion of an example of an embodiment of a TFDF filter 310 which may be used is illustrated as having tortuous paths 312 therethrough, with settling zones 314 in which intermediate particles may be captured, and narrower channels 316 through which smaller particles may pass to outside the filter 310, in a manner known in the art of TFDF filters. In some further examples of such embodiment, the TFDF filter has a pore size for removing particles such as cell debris of about 2-5 μm in diameter. The secondary filtration system 300 may be operated in a TFF mode, or, more particularly, an ATF mode.

In a cell culture system 100 with a microfilter in the primary filtration system 200, the secondary filtration system 300 may be run intermittently for rapid removal of cell debris and/or for harvesting desired products (e.g., protein). For instance, the secondary filtration system 300 with the TFDF filter may be turned on intermittently when cell debris content has increased in the system (e.g., as may be detected by a sensor or other monitor in the process vessel 110) and may be fouling the microfilter in the primary filtration system 200. Such intermittent use of the secondary filtration system 300 allows cells to grow healthily in the cell culture system 100 without being adversely impacted by cellular waste products being produced during cellular growth. It will be appreciated that the running of the secondary filtration system 300 may be process dependent. In other words, in some instances, when cell debris content is high in the bioreactor, only the secondary filtration system 300 will be running to entrap/remove that debris, and in other cases, both filtration systems 200, 300 will be running, with a portion of the cell culture volume being filtered through the secondary filtration system 300, depending on the filter performance.

In some further examples of an embodiment of a batch run cell culture system 100 with a microfilter in the primary filtration system 200, the secondary filtration system 300 may be turned on a percentage of the time less than 50% of the time the primary filtration system 200 is on. For instance, the secondary filtration system 300 may be turned on for a few hours (e.g., 2-6 hours) per day, or a few days (e.g., 1-3 days) per week, depending on how the rate of formation of cell debris (e.g., cell lysis) in the cell culture system 100. In some aspects, the secondary filtration system 300 may run at a circulation rate of 1000-4000 shear s⁻¹, and fluxes of approximately 50 liters per meter² per hour up to approximately 2000 liters per meter² per hour. In some instances, one to two vessel volume exchanges in such embodiment of a cell culture system 100 may result in 66-88% removal of cell debris. The volumetric throughput of such embodiment can be from approximately 5000 liters per meter² up to at least (or greater) than approximately 20,000 liters per meter² for a 0.6 m² filter, thus allowing for two vessel volume exchanges up to eight or more vessel volume exchanges (e.g., for a 1500 L vessel), depending on the cell culture conditions in the channel spacers 130. The operating flux for the secondary filtration system 300 may be high, such as 500-2000 liters per meter² per hour. As may be appreciated, such embodiment allows faster volume exchanges and quicker removal of cell debris from the bioreactor than achieved by previous systems. Removal of cell debris including the DNA, protein and lipids will improve the life of the perfusion filter. Any product that is removed with the TFDF can be recovered from the final pool to keep product loss at a minimum.

In another example of an embodiment of a cell culture system 100, the primary filtration system 200 uses a microfilter, in a TFF system, or an ATF system, as in the previously-described example of an embodiment, but the cell culture system 100 is, instead, run in continuous mode. In such embodiment, the secondary filtration system 300 has a TFDF filter which may be used for harvesting purposes as well, but with less cell culture flowing through the secondary filtration system 300 than through the primary filtration system 200. For instance, more than half the harvest flow (the flow of cell culture 112 out of the process vessel 110 and into a filter) may be directed to the primary filtration system 200 and less than half of the harvest flow may be directed to the secondary filtration system 300. In some embodiments, approximately 80% of the harvest flow is directed through the microfilter of the primary filtration system 200, with the remainder) of the harvest flow (approximately 20%) being directed through the TFDF filter of the secondary filtration system 300. Such amounts of harvest flow through each filtration system 200, 300 are adjustable and/or optimizable based on any of a variety of process conditions as appreciated by those of ordinary skill in the art.

In the above example of a continuous mode cell culture system 100 with a microfilter in the primary filtration system 200 and a TFDF filter in the secondary filtration system 300, the microfilter in the primary filtration system 200 is used for media exchange to maintain the healthiness of the cell culture 112 and for sieving protein (i.e., harvesting cell products). Additionally, simultaneously filtering a portion of the cell culture 112 through the TFDF filter in the secondary filtration system 300 permits much higher protein sieving to occur and thus increase the rapidity of protein harvesting and ultimately the cumulative protein yields. The secondary filtration system 300 will also act to increase the longevity of the filters in the primary filtration system 200 by removing cellular debris with greater efficiency and/or efficacy than previously possible, allowing the cells in the cell culture 112 to remain alive and to continue to grow for a longer time, thus allowing for greater protein production by the cell culture system 100.

In yet another example of an embodiment, the cell culture system 100 is run in semi-batch mode, as in the first example of an embodiment described above. However, instead of a microfilter, the primary filtration system 200 may use an ultrafilter. As such, instead of filtering to harvest products (e.g., proteins), the primary filtration system 200 is used for media exchange and/or to filter out waste products and/or to filter out spent media. Similar to the first example of an embodiment described above, the secondary filtration system 300 includes a TFDF filter which may be turned on intermittently (e.g., every 1-2 or 1-3 days in a week, or 3-4 days in a week, depending on the protein production and target desired concentration). The primary ultrafilter may be turned off while the TFDF is turned on temporarily to harvest the batch of protein. In this configuration, the protein that is accumulated in the bioreactor can be harvested using TFDF. In this example, the TFDF is not only eliminating the cell debris but also harvesting the protein. The processing duration for TFDF filters can be adjusted based on the process parameters and protein concentrations in the bioreactor. After the harvesting step performed by the TFDF, the ultrafilter may be turned on again to accumulate another batch of protein. The combination of a system using an ultrafilter and a TFDF system increases the volumetric production of the target protein.

It will be appreciated that because of the ultrafilter that is used in this example of an embodiment, the volumetric production of the protein is increased. Using the secondary filtration system 300, and, particularly, a TFDF filter in batch mode expected to significantly increases volumetric productivity. Productivity can be further optimized and adjusted to ease up the downstream processes and column capturing. The combination of a system using an ultrafilter along with a TFDF system allows for harvesting protein when it reaches a desired concentration. The harvest concentration can be selected based on the existing downstream tools making the entire process economical.

In accordance with one aspect of an example of an embodiment of a batch run cell culture system 100 with an ultrafilter in the primary filtration system 200, the secondary filtration system 300 with a TFDF filter may be operated in an ATF mode as it may be turned on every 1-2 days in a week or 3-4 days in a week depending on the protein production and target desired concentrations. Such configuration may have a higher yield than a continuous batch configuration, which generally would otherwise have a higher yield than a standard batch mode. In the case of a concentrated fed-batch, the entire batch is harvested only once, and the final harvest concentration might vary depending on the cell growth and batch-to-batch variation. On the contrary, in an approach in accordance with various principles of the present disclosure, the harvest process can be implemented when the desired protein concentration is achieved. Since the process can be implemented several times in the same vessel, the process yield and volumetric productivity will be higher than a typical concentrated fed-batch.

It will be appreciated that although the primary filtration system 200 and/or secondary filtration system 300 of any of the above-described examples of embodiments may be run in ATF mode, the primary filtration system 200 and/or secondary filtration system 300 may alternatively be run in TFF mode instead, with benefits over prior cell culture systems.

The TFDF filters used in the present disclosure may be any suitable TFDF filter, such as disclosed in U.S. Pat. No. 10,538,727 to Bransby et al., issued Jan. 21, 2020, and titled Tangential Flow Depth Filtration Systems And Methods Of Filtration Using Same; or U.S. Patent Application Publication US2014/0093952 to Serway, published Apr. 3, 2014, and titled Method For Proliferation Of Cells Within A Bioreactor Using A Disposable Pumphead And Filter Assembly; or U.S. Pat. No. 10,711,238 to Serway, issued Jul. 14, 2020, and titled Method For Proliferation Of Cells Within A Bioreactor Using A Disposable Pumphead And Filter Assembly; or Published International Patent Application Number WO2017/180573, published Oct. 19, 2017, and titled Thick Wall Hollow Fiber Tangential Flow Filter, all of which applications are incorporated herein in their entireties for all purposes. It will be appreciated that TFDF fibers used in the present disclosure may have a wide range of lengths. In some embodiments, the hollow fibers may have a length of at least about 200 mm and at most about 2000 mm in length, among other values. The TFDF fibers may be formed from a variety of materials using a variety of processes. For example, the TFDF fibers may be formed by assembling numerous particles, filaments, or a combination of particles and filaments into a tubular shape. In some embodiments, melt-blown filaments, such as described, for example, in U.S. Pat. No. 5,607,766 to Berger, issued Mar. 4, 1997, and titled Polyethylene terephthalate sheath/thermoplastic polymer core biocomponent fibers, method of making same and products formed therefrom, may be used. The pore size and distribution of hollow fibers formed from particles and/or filaments will depend on the size and distribution of the particles and/or filaments that are assembled to form the hollow fibers. The pore size and distribution of hollow fibers formed from filaments will also depend on the density of the filaments that are assembled to form the hollow fibers. For example, mean pore sizes of at least about 0.5 microns and at most about 50 microns may be created by varying filament density. The wall of the TFDF filter may have a density (a percentage volume that the filaments take up compared to an equivalent solid volume of the polymer) produced to correlate with the amount of a variable cell density at which the filter can operate without fouling. In certain embodiments, the hollow fiber may be further coated with a suitable coating material (e.g., PVDF) either on the inside or outside of the fiber, which coating process may also act to reduce the pore size of the hollow fiber, if desired. It will be appreciated that TFDF fibers may have various other novel applications not disclosed in the above-referenced patent documents incorporated by reference herein.

It should be understood that, as described herein, an “embodiment” (such as illustrated in the accompanying Figures) may refer to an illustrative representation of an environment or article or component in which a disclosed concept or feature may be provided or embodied, or to the representation of a manner in which just the concept or feature may be provided or embodied. However illustrated embodiments are to be understood as examples (unless otherwise stated), and other manners of embodying the described concepts or features, such as may be understood by one of ordinary skill in the art upon learning the concepts or features from the present disclosure, are within the scope of the disclosure. In addition, it will be appreciated that while the Figures may show one or more embodiments of concepts or features together in a single embodiment of an environment, article, or component incorporating such concepts or features, such concepts or features are to be understood (unless otherwise specified) as independent of and separate from one another and are shown together for the sake of convenience and without intent to limit to being present or used together. For instance, features illustrated or described as part of one embodiment can be used separately, or with one or more other features to yield a still further embodiment. Thus, it is intended that the present subject matter covers such modifications and variations as come within the scope of the appended claims and their equivalents. It is to be understood by one of ordinary skill in the art that the present discussion is a description of illustrative examples of embodiments only, and is not intended as limiting the broader aspects of the present disclosure.

It will be appreciated features described with respect to one embodiment typically may be applied to another embodiment, whether or not explicitly indicated. The various features hereinafter described may be used singly or in any combination thereof. Therefore, the present invention is not limited to only the embodiments specifically described herein.

The foregoing discussion has broad application and has been presented for purposes of illustration and description and is not intended to limit the disclosure to the form or forms disclosed herein. It will be understood that various additions, modifications, and substitutions may be made to embodiments disclosed herein without departing from the concept, spirit, and scope of the present disclosure. In particular, it will be clear to those skilled in the art that principles of the present disclosure may be embodied in other forms, structures, arrangements, proportions, and with other elements, materials, and components, without departing from the concept, spirit, or scope, or characteristics thereof. For example, various features of the disclosure are grouped together in one or more aspects, embodiments, or configurations for the purpose of streamlining the disclosure. However, it should be understood that various features of the certain aspects, embodiments, or configurations of the disclosure may be combined in alternate aspects, embodiments, or configurations. While the disclosure is presented in terms of embodiments, it should be appreciated that the various separate features of the present subject matter need not all be present in order to achieve at least some of the desired characteristics and/or benefits of the present subject matter or such individual features. One skilled in the art will appreciate that the disclosure may be used with many modifications or modifications of structure, arrangement, proportions, materials, components, and otherwise, used in the practice of the disclosure, which are particularly adapted to specific environments and operative requirements without departing from the principles or spirit or scope of the present disclosure. For example, elements shown as integrally formed may be constructed of multiple parts or elements shown as multiple parts may be integrally formed, the operation of elements may be reversed or otherwise varied, the size or dimensions of the elements may be varied. Similarly, while operations or actions or procedures are described in a particular order, this should not be understood as requiring such particular order, or that all operations or actions or procedures are to be performed, to achieve desirable results. Additionally, other implementations are within the scope of the following claims. In some cases, the actions recited in the claims can be performed in a different order and still achieve desirable results. The presently disclosed embodiments are therefore to be considered in all respects as illustrative and not restrictive, the scope of the claimed subject matter being indicated by the appended claims, and not limited to the foregoing description or particular embodiments or arrangements described or illustrated herein. In view of the foregoing, individual features of any embodiment may be used and can be claimed separately or in combination with features of that embodiment or any other embodiment, the scope of the subject matter being indicated by the appended claims, and not limited to the foregoing description.

In the foregoing description and the following claims, the following will be appreciated. The phrases “at least one”, “one or more”, and “and/or”, as used herein, are open-ended expressions that are both conjunctive and disjunctive in operation. The terms “a”, “an”, “the”, “first”, “second”, etc., do not preclude a plurality. For example, the term “a” or “an” entity, as used herein, refers to one or more of that entity. As such, the terms “a” (or “an”), “one or more” and “at least one” can be used interchangeably herein. All directional references (e.g., proximal, distal, upper, lower, upward, downward, left, right, lateral, longitudinal, front, back, top, bottom, above, below, vertical, horizontal, radial, axial, clockwise, counterclockwise, and/or the like) are only used for identification purposes to aid the reader's understanding of the present disclosure, and/or serve to distinguish regions of the associated elements from one another, and do not limit the associated element, particularly as to the position, orientation, or use of this disclosure. Connection references (e.g., attached, coupled, connected, and joined) are to be construed broadly and may include intermediate members between a collection of elements and relative movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority, but are used to distinguish one feature from another.

The following claims are hereby incorporated into this Detailed Description by this reference, with each claim standing on its own as a separate embodiment of the present disclosure. In the claims, the term “comprises/comprising” does not exclude the presence of other elements, components, features, regions, integers, steps, operations, etc. Additionally, although individual features may be included in different claims, these may possibly advantageously be combined, and the inclusion in different claims does not imply that a combination of features is not feasible and/or advantageous. In addition, singular references do not exclude a plurality. Reference signs in the claims are provided merely as a clarifying example and shall not be construed as limiting the scope of the claims in any way.

What is claimed is:

1. A method of improving volumetric productivity from a cell culture, said method comprising:
(a) filtering a cell culture through an ultrafilter or a microfilter operating in tangential flow filtration mode or alternating flow filtration mode; and
(b) filtering the cell culture concurrently or intermittently through a tangential flow depth filtration system to remove cellular debris and/or to harvest cell product.

2. The method of claim 1, wherein said cell culture is a perfusion or concentrated fed-batch cell culture, and wherein filtering in step (a) uses a microfilter operating in tangential flow filtration mode or alternating tangential filtration mode.

3. The method of claim 1, wherein said cell culture is a concentrated fed-batch cell culture, and wherein filtering in step (a) uses an ultrafilter operating in tangential flow filtration or alternating tangential filtration mode.

4. The method of claim 1, wherein filtering the cell culture through a tangential flow depth filtration system prolongs the usable life of the ultrafilter or microfilter.

5. A system for filtering biological materials, said system comprising:
a primary filtration system; and
a secondary filtration system;
wherein said secondary filtration system comprises a tangential flow depth filtration filter.

6. The system of claim 5, wherein said primary filtration system comprises a microfilter or an ultrafilter.

7. The system of claim 5, wherein said system for filtering biological materials produces a cell culture utilizing a batch production process.

8. The system of claim 5, wherein said primary filtration system is on continuously and said secondary filtration system is turned on continuously or intermittently.

9. The system of claim 5, wherein said system for filtering biological materials produces a cell culture utilizing a continuous production process.

10. The system of claim 9, wherein greater than 50% of the volume of the cell culture is filtered by said primary filtration system and less than 50% of the volume of the cell culture is filtered by said secondary filtration system.

11. The system of claim 10, wherein 80% of the volume of the cell culture is filtered by said primary filtration system and 20% of the volume of the cell culture is filtered by said secondary filtration system.

12. The system of claim 5, wherein said primary filtration system comprises a microfilter and said system for filtering biological materials produces a cell culture utilizing a batch production process.

13. The system of claim 5, wherein said primary filtration system comprises a microfilter and said system for filtering biological materials produces a cell culture utilizing a continuous production process.

14. The system of claim 5, wherein said primary filtration system comprises an ultrafilter and said system for filtering biological materials produces a cell culture utilizing a batch production process.

15. The system of claim 5, wherein said primary filtration system is on continuously and said secondary filtration system is turned on intermittently.

16. The system of claim 5, wherein flow volume to said secondary filtration system is less than the flow volume to said primary filtration system.

17. The system of claim 5, wherein said primary filtration system is an alternating tangential flow filtration system.

18. The system of claim 5, further comprising a process vessel.

19. A method of harvesting a cell product from a cell culture, said method comprising:
(a) harvesting the cell product from a perfusion or concentrated fed-batch cell culture using a microfilter operating in tangential flow filtration or alternating tangential flow mode into a first permeate;
(b) removing cellular debris and further harvesting the cell product by concurrently or intermittently filtering the cell culture using a tangential flow depth filtration filter operating in tangential flow filtration or alternating tangential flow mode into a second permeate; and
(c) recovering the cell product from the first and second permeates.

20. The method of claim 19, wherein the cell culture is a perfusion or concentrated fed-batch cell culture.
